(19) United States
(12) Patent Application Publication
(10) Pub. No.: US 2010/0086574 A1
(43) Pub. Date: Apr. 8, 2010

(54) Use of aqueous emulsions in the form of foam for the reloading of textiles

(75) Inventors: Raymond Mathis, Duesseldorf (DE); Robert Schutz, Tonisvorst (DE); Jurgen Falkowski, Monheim (DE); Werner Mauer, Albersloh (DE)

Correspondence Address: FOX ROTHSCHILD LLP, 997 Lenox Drive, Bldg. #3, Lawrenceville, NJ, 08648, US

(73) Assignee: COGNIS IP MANAGEMENT GMBH, Duesseldorf (DE)

(21) Appl. No.: 12/522,157

(22) PCT Filed: Dec. 8, 2007
(86) PCT No.: PCT/EP2007/010714

§ 371 Date: Jul. 3, 2009

(30) Foreign Application Priority Data
Jan. 4, 2007 (DE) 10 2007 001 115.8

(51) Int. Cl. A61K 8/11

(52) U.S. Cl. 424/401

(57) ABSTRACT

The invention relates to the use of aqueous emulsions and/or dispersions in the form of foam for the reloading of textiles. These aqueous emulsions and/or dispersions contain (a) 0-25% by weight of one or several skin-protecting oils, (b) 0-25% by weight of microcapsules loaded with active ingredients, (c) 0-20% by weight of one or several emulsifiers, (d) 0.1-25% by weight of one or several foam-producing agents, (e) 0-10% by weight of one or several foam stabilizers, and (f) the remainder at 100% by weight of water, with the condition that at least one of the components (a) or (b) must be present in a quantity of more than 0% by weight, and the further condition that the foam must fulfill the following conditions: the foam density is in the range of 50 to 300 g/l and the foam disintegration time is in the range of 2 to 30 min.

### FIELD OF THE INVENTION

The invention relates to the use of aqueous emulsions in foam form for the reloading of clothing textiles.

### PRIOR ART

For the finishing of high-value textiles, oil mixtures are being used more and more often which impart skincare properties to the textiles. When absorbed over the fabric, these oil mixtures can impart, for example, moisturizing, smoothing, cooling, warming or refatting properties. For the first finishing of textiles with oil mixtures, an aqueous emulsion of these oil mixtures, which is further diluted in the textile liquor, is usually used. These aqueous solutions can then be used, for example, in a padding or absorption method for the finishing of textiles. However, after a few washing operations, the effect of such a first finishing deteriorates.

For this reason there is a need for methods which are easy to carry out which return the particular properties of the first finishing to the textile.

DE-A-102005045138 describes aqueous microcapsule dispersions comprising a) water, b) microcapsules which are loaded with one or more ingredients and/or active ingredients, and c) polymeric dispersants, where these polymers may be homopolymers or copolymers and where these polymers are composed of at least 5 monomer building blocks. These dispersions according to the invention are storage-stable in the long term. These microcapsule dispersions can be used for reloading applications.

DE-A-102005059721 describes a method for the finishing of textiles with care oils, where aqueous emulsions with a viscosity below 200 mPas (measured according to Brookfield at 20° C.) comprising (a) water, (b) one or more care oils and (c) one or more emulsifiers are applied to textiles, with the proviso that the aqueous emulsion is applied to the textile by spraying. However, this method has the disadvantage that during spraying at least a certain amount of the emulsion lands aside from the textile ware to be treated and is lost.

### DESCRIPTION OF THE INVENTION

Within the context of the present invention, reloading is understood as meaning a reloading of textiles (for example after washing operations). Reloading therefore does not refer to an industrial first finishing of textiles, but refers to the textile being refinished with skincare oils and/or active ingredients. Depending on its nature, such reloading is carried out in particular by an end consumer.

It was an object of the present invention to develop a method which can be used easily and gives back to the textile the particular properties of the first finishing with regard to skincare oils and/or active ingredients. It was a further object to provide aqueous emulsions and/or dispersions with a content of skincare oils and/or active ingredients, where these emulsions and/or dispersions should be able to be readily foamed with air and form a stable reload foam which can be worked in—spreadably and stain free—readily and evenly on a textile—in particular items of clothing which are worn directly against the skin, for example underwear, trousers, shirts and stockings. The reload foams should be able to be readily incorporated into the textile homogeneously and without spotting. In this connection, a rapid processability should be ensured. For this, it is necessary that the foam on the one hand has adequate stability to be able to be incorporated into the textile without disintegrating and beforehand giving rise to spotting and on the other hand the foam should not be so stable that incorporation into the textile is not possible or is possible only with considerable time expenditure. In particular, the reload foam should be able to be incorporated in the stated manner into textiles which are still damp from spinning, as are present after the washing and spinning of textiles.

Particularly, it may be established that within the context of the present invention, textiles is used to mean items of clothing.

The present invention provides the use of aqueous emulsions and/or dispersions in foam form comprising
a) 0-25% by weight of one or more skincare oils,
b) 0-25% by weight of microcapsules loaded with active ingredients,
c) 0-20% by weight of one or more emulsifiers,
d) 0.1-25% by weight of one or more foam-producing agents,
e) 0-10% by weight of one or more foam stabilizers and
f) remainder to 100% by weight of water,
   with the proviso that at least one of components a) and b) must be present in an amount of more than 0% by weight,
   and the further proviso that the foam must satisfy the following conditions:
   foam density in the range from 50 to 300 g/l,
   foam disintegration time in the range from 2 to 30 min,
      for the reloading of textiles.

The % by weight data of the individual components are always based on the entire emulsion and/or dispersion—this applies generally for the present invention.

The present invention further provides a method for the reloading of textiles, where an aqueous emulsion and/or dispersion comprising
a) 0-25% by weight of one or more skincare oils,
b) 0-25% by weight of microcapsules loaded with active ingredients,
c) 0-20% by weight of one or more emulsifiers,
d) 0.1-25% by weight of one or more foam-producing agents,
e) 0-10% by weight of one or more foam stabilizers and
f) remainder to 100% by weight of water,
   with the proviso that at least one of components a) and b) must be present in an amount of more than 0% by weight,
   is converted to foam form, with the proviso that the foam must satisfy the following conditions:
   foam density in the range from 50 to 300 g/l,
   foam disintegration time in the range from 2 to 30 min.

The present invention further provides compositions for the reloading of textiles, where these compositions are aqueous emulsions and/or dispersions comprising
a) 0-25% by weight of one or more skincare oils,
b) 0-25% by weight of microcapsules loaded with active ingredients,
c) 0-20% by weight of one or more emulsifiers,
d) 0.1-25% by weight of one or more foam-producing agents,
e) 0-10% by weight of one or more foam stabilizers and
f) remainder to 100% by weight of water,
   with the proviso that at least one of components a) and b) must be present in an amount of more than 0% by weight,
   and the additional proviso that upon converting the emulsion and/or dispersion into a foam, which takes place by foaming with a gas, the foam must satisfy the following conditions:
   foam density in the range from 50 to 300 g/l,
   foam disintegration time in the range from 2 to 30 min.

The present invention further provides a system comprising a manually operable foam dispenser and an aqueous emulsion and/or dispersion comprising
a) 0-25% by weight of one or more skincare oils,
b) 0-25% by weight of microcapsules loaded with active ingredients,
c) 0-20% by weight of one or more emulsifiers,
d) 0.1-25% by weight of one or more foam-producing agents,
e) 0-10% by weight of one or more foam stabilizers and
f) remainder to 100% by weight of water,
   with the proviso that at least one of components a) and b) must be present in an amount of more than 0% by weight,
   and the further proviso that the foam which emerges upon operating the foam dispenser must satisfy the following conditions:
   foam density in the range from 50 to 300 g/l,
   foam disintegration time in the range from 2 to 30 min.

The present invention further provides a system comprising a compressed-gas-operated foam dispenser and an aqueous emulsion and/or dispersion comprising
a) 0-25% by weight of one or more skincare oils,
b) 0-25% by weight of microcapsules loaded with active ingredients,
c) 0-20% by weight of one or more emulsifiers,
d) 0.1-25% by weight of one or more foam-producing agents,
e) 0-10% by weight of one or more foam stabilizers and
f) remainder to 100% by weight of water,
   with the proviso that at least one of components a) and b) must be present in an amount of more than 0% by weight,
   and the further proviso that the foam which emerges upon operating the foam dispenser must satisfy the following conditions:
   foam density in the range from 50 to 300 g/l,
   foam disintegration time in the range from 2 to 30 min.

The reload foams to be used according to the invention may be emulsions or dispersions. The emulsions may be conventionally prepared O/W emulsions (“macroemulsions”) or else the so-called PIT or micro- or nanoemulsions known to the person skilled in the art.

Preferred textiles which are finished with the reload foams according to the invention are items of clothing of any type, preferably those which are worn directly against the skin, for example underwear, trousers, shirts and stockings.

The foams to be used according to the invention are defined by the two aforementioned physicochemical parameters. These may be explained as follows:

Foam density (FD) is to be understood as meaning the quotient of foam weight (in grams) and foam volume (in liters) at a temperature of 20° C. The FD is given in g/l. Example: 2 kg of an aqueous liquor are processed to give 8 liters of foam. The FD is then 2000 g/8 l=250 g/l. Within the context of the present invention, the FD is preferably in the range from 50 to 250 g/l and in particular in the range from 100 to 200 g/l.

Foam decomposition time (FT) is to be understood as meaning the time in which, at 20° C., from a foam volume of 1 liter, the liquid present therein has flowed out in an amount of 50 ml. The FT is a measure of the durability of the foam. The FT is determined as follows: a graduated conical 1 liter sedimentation funnel (in accordance with DIN 12672-K) is filled with foam up to the 1000 ml mark over the course of 60 s. The foam is produced here from the aqueous emulsion or dispersion by means of a pump foam bottle. Then, using a stopwatch, the time is measured in which 50 ml of liquid settle out.

The pump foam bottle used here is a commercially available manually operable mechanical foam dispenser from Rexam Airspray International BV (bottle 200 ml HDPE—BE-43C1-200HE-10002; foamer pump white —F202.093.0001).

Within the context of the present invention, the FT is preferably in the range from 5 to 20 minutes and in particular in the range from 7 to 18 minutes.

As already stated above, both the foam decomposition time and the foam density are determined at a temperature of 20° C.

If desired, the reload foam to be used according to the invention can also comprise further constituents. Examples thereof are viscosity regulators and vegetable extracts which can be used in aqueous or nonaqueous form. In the case of aqueous plant extracts, it is usually possible to dispense with the use of special emulsifiers for these plant extracts. Examples of suitable plant extracts are Aloe vera, horse chestnut extract, etc. Within the context of the present invention, aqueous plant extracts are preferably used in an amount of from 0 to 5%.

### Foam Dispenser

In one preferred embodiment, the reload foam to be used according to the invention is applied to the textile ware to be finished using a manually operable mechanical foam dispenser with pump mechanism. For this purpose it is possible to use per se any desired foam dispenser of this type with a pump mechanism. The reload emulsions or reload dispersions according to the present invention are foamed with air from a dispenser. Dispensers based on propellant gas, however, are not used within the context of the present invention.

The foam dispenser to be used allows the user to convert in a simple manner the aqueous reload emulsions or dispersions according to the invention into a particular reload foam which fulfils the aforementioned complex task. The foam dispenser should be designed so that it has a mixing ratio of air to liquid (reload emulsion or dispersion) such that the reload foam produced satisfies the two aforementioned conditions, namely:
foam density in the range from 50 to 300 g/l,
foam decomposition time in the range from 2 to 30 min.

One example of a suitable foam dispenser is the foam dispenser with pump mechanism from Airspray International B.V., as is described in WO-A-03/088941 on page 25 ff and FIG. 1. Suitable foam dispensers can also be acquired, for example, from Microtec Labs Inc. (5747 Executive Bvld., Dayton, Ohio 45424, USA).

The reload foam to be used according to the invention is applied directly to the fabric and then massaged in. After a drying time, which depends on the type of fabric, the fabric then has again the original skincare properties and can then be put on immediately. The skincare effect can be controlled in the case of the reload foam according to the invention via the applied amount of care oils and/or active ingredients. Besides the skincare properties, the foam can impart a better feel and/or better smoothness to the textile, which simplifies, for example, the putting on of close-fitting items of clothing (e.g. compression stockings).

The compounds c) (emulsifiers), d) (foam-producing agents) and e) (foam stabilizers) are defined by their function. This means:
any substance which is able to convert an aqueous system with a content of the components a) (skincare oils) and/or b) (microcapsules loaded with active ingredients) into an emulsion and/or dispersion is referred to—irrespective of the substance class—as emulsifier c).
any substance which is able to convert an aqueous emulsion and/or dispersion with a content of the components a) (skincare oils) and/or b) (micro-capsules loaded with active ingredients) and c) (emulsifiers) into a foam upon foaming with air is referred to—irrespective of the substance class—as foam-producing agent d).
any substance which is able to stabilize a foam which is formed upon foaming an aqueous emulsion and/or dispersion with a content of the components a) (skincare oils) and/or b) (microcapsules loaded with active ingredients) and c) (emulsifiers) with air against rapid decomposition is referred to—irrespective of the substance class—as foam stabilizer e).

It is directly clear that the compounds c), d) and e) can overlap in structural terms. For example, in one embodiment, the compounds c), d) and e) are selected from the group of surfactants.

Here, in a first embodiment, another substance is used for each of the compounds c), d) and e).

It may, however, also be a different embodiment that a surfactant fulfils a double function, i.e. is
i) simultaneously emulsifier and foam-producing agent or
ii) simultaneously emulsifier and foam stabilizer or
iii) simultaneously foam-producing agent and foam stabilizer.

In rare cases, it may be that a surfactant is used which is
iv) simultaneously emulsifier, foam-producing agent and foam stabilizer.

In relation to the stated quantitative ranges for the compounds c), d) and e), the embodiments i) to iv) mean:
In case i), the quantitative range arises for the surfactant which is simultaneously emulsifier and foam-producing agent by adding the values given for c) and d), where in each case the lower limits and the upper limits are to be added.
In case ii), the quantitative range arises for the surfactant which is simultaneously emulsifier and foam stabilizer by adding the values given for c) and e), where in each case the lower limits and the upper limits are to be added.
In case iii), the quantitative range arises for the surfactant which is simultaneously foam-producing agent and foam stabilizer by adding the values given for d) and e), where in each case the lower limits and the upper limits are to be added.
In case iv), the quantitative range arises for the surfactant which is simultaneously emulsifier, foam-producing agent and foam stabilizer by adding the values given for c), d) and e), where in each case the lower limits and the upper limits are to be added.

### Compounds a)

As already stated, component (a) is skincare oils. Here, the term “oil” is not to be understood in the chemically restricted sense of “triglyceride”. Rather, oil is to be understood as meaning a component which has an oily consistency at room temperature. Component (a) is preferably selected from the group of mono-glycerides, diglycerides, triglycerides and fatty acid alkyl esters. These may either be substances of natural origin or else synthetic substances.

In one embodiment, the oils (a) function not only themselves as skincare substances, but they can moreover also comprise further oil-soluble skincare substances in dissolved form.

The compounds a) are used in an amount of from 0 to 25% by weight, preferably 3 to 20% by weight and in particular 5 to 15% by weight. Examples of particularly suitable oils a) are, for example, coconut oil, squalane, jojoba oil, Shea butter, vitamin E, myritol 318, cetiol SN, paraffins, white oils, dimethyl-siloxane.

### Microcapsules b)

Within the context of the present inventions, microcapsules are in principle to be understood as meaning organic polymers with a certain three-dimensional structure (cf. in this regard: K. Lacasse and W. Baumann, Textile Chemicals, Environmental Data and Facts, Berlin 2004, pages 468-482). As regards the three-dimensional structure, this is hollow bodies which typically have a diameter in the range from 2 to 2000 μm and an external diameter in the range from 0.1 to 200 μm and in particular 0.5 to 150 μm. On account of this hollow-body structure, the microcapsules can be loaded with ingredients and/or active ingredients.

Within the context of the present invention, loaded microcapsules are always used, i.e. microcapsules which are loaded with one or more ingredients and/or active ingredients. Suitable ingredients and/or active ingredients are in principle all substances which are to pass onto the skin upon wearing the textile which has been finished with the loaded microcapsules. These may be, for example, fats, oils, plant extracts, vitamins, fragrances, repellents, insecticides and the like. The oils are preferably vegetable oils with skincare and health-promoting properties, for example coconut oil, passionflower oil, Shea butter, rosehip kernel oil, lavender oil, apricot kernel oil. The plant extracts are preferably algae extracts such as Rhodysterol, Herbalia centella, Herbalia green tea, Herbalia horse chestnut and Aloe vera. (All Herbalia grades are available from Cognis).

Of particular importance within the context of the present invention are those active ingredients and/or ingredients which have the following properties: skincare, moisturizing, stimulating, calming, cellulite-reducing, skin-tightening, repelling, refreshing, warming, stimulating.

The encapsulated substances, also called core material below, can consist of any desired solid, liquid or gaseous materials which are to be incorporated in encapsulated form into corresponding products. Preferably, the core materials used are fragrances, such as perfume oils, or for the respective field of use, care substances.

Perfume oils and/or fragrances which can be used are individual odorant compounds, e.g. the synthetic products of the ester, ether, aldehyde, ketone, alcohol and hydrocarbon types. Odorant compounds of the ester type are e.g. benzyl acetate, phenoxyethyl isobutyrate, p-tert-butylcyclohexyl acetate, linalyl acetate, dimethylbenzyl carbonyl acetate, phenylethyl acetate, linalyl benzoate, benzyl formate, ethylmethylphenyl glycinate, allyl cyclohexylpropionate, styrallyl propionate and benzyl salicylate. The ethers include, for example, benzyl ethyl ether, the aldehydes include e.g. the linear alkanals having 8-18 carbon atoms, citral (geranial), citronellal, citronellyloxyacetaldehyde, cyclamenaldehyde, hydroxycitronellal, lilial and bourgeonal. The ketones include e.g. the ionones, α-isomethylionone and methyl cedryl ketone, the alcohols include anethole, citronellol, eugenol, geraniol, linalool, phenylethyl alcohol and terpineol, the hydrocarbons include primarily the terpines such as limonene and α-pinene. Eucalyptol (1,8-cineol) can also be used as fragrance. However, preference is given to using mixtures of different odorants which together produce a pleasing scent note. Such perfume oils can also comprise natural odorant mixtures, as are accessible from plant sources, e.g. pine oil, citrus oil, jasmine oil, patchouli oil, rose oil or ylang ylang oil. Likewise suitable are clary sage oil, camomile oil, oil of cloves, melissa oil, mint oil, eucalyptus oil, cinnamon leaf oil, linden blossom oil, juniper berry oil, vetiver oil, olibanum oil, galbanum oil and labdanum oil and also orange blossom oil, neroliol, orange peel oil and sandalwood oil.

Moreover, nitriles, sulfides, oximes, acetals, ketals, acids, Schiff's bases, heterocyclic nitrogen compounds such as indole and quinoline, pyrazines, amines such as anthranilates, amides, organohalogen compounds such as rose acetate, nitrated compounds such as nitromusk, heterocyclic sulfur compounds such as thiazoles and heterocyclic oxygen compounds such as epoxides, which are all known to the person skilled in the art as possible odorants, can be used.

Examples of care components are vitamins and provitamins, such as vitamin A, vitamin C, vitamin E (α-tocopherol), vitamin F (polyene fatty acids), panthenol (provitamin B5), betacarotene (provitamin A) and derivatives thereof (e.g. esters such as stearyl ascorbate), plant extracts, biopolymers, antidandruff agents, UV protectants, emollients (cosmetic oils), silicone oils.

In the case of cosmetic applications, preferred care components are tocopherols and their lipid-soluble derivatives. Suitable tocopherols are e.g. the natural tocopherols and their mixtures, and also synthetic tocopherols. Suitable derivatives are e.g. tocopheryl acetate, tocopheryl nicotinate, tocopheryl ascorbate, tocopheryl retinoate, tocopheryl succinate, tocopheryl linoleate or tocopheryl benzoate.

The microcapsules b) are used in an amount of from 0 to 25% by weight, preferably 3 to 25% by weight and in particular 15 to 25% by weight.

### Compounds c)

As already stated, the compounds (c) are emulsifiers. These serve to emulsify and/or to disperse the care oils (a) in water. The selection of emulsifiers per se is not subject to particular restrictions.

The emulsifiers (c) may be classic synthetic emulsifiers, such as surfactants—for example ethoxylated fatty alcohols—or natural emulsifiers, such as, for example, lecithin. Here, emulsifiers with an HLB value in the range from 8 to 18 are preferred.

If PIT emulsions are used, special emulsifier mixtures, such as, for example, Emulgade SE-PF (manufacturer: Cognis) are preferably used.

In one embodiment, the compounds c) used are polymeric emulsifiers, i.e. compounds which are structurally described as polymers and which develop an emulsifying effect with regard to the care oils b). Examples of polymeric emulsifiers c) whose monomer building blocks are of natural origin are, for example, polymers based on cellulose (e.g. Na carboxymethylcellulose) or polysaccharides (e.g. xanthan gum, gellan gum, guar or pectins). Examples of polymeric emulsifiers c) whose monomer building blocks are of synthetic origin are, for example, acrylates (e.g. Na polyacrylates), methacrylates or alkyl acrylates (e.g. Pemulen). If desired, the monomer building blocks of which the emulsifiers c) are composed can also be chemically modified. In a very particularly preferred embodiment, the polymeric emulsifiers c) used are compounds which are selected from the group xanthan gum, gellan gum, guar, polyacrylates.

These emulsifiers can be used individually or in a mixture with one another. The emulsifiers c) are used in an amount of from 0 to 20% by weight, preferably 0 to 15% by weight and in particular 0 to 10% by weight.

### Compounds d)

As already stated, the compounds d) are foam-producing agents. In this regard, there are no restrictions per se, although surfactants are preferably used. In particular, readily foaming anionic, nonionic, cationic or amphoteric surfactants or mixtures thereof are used. The compounds d) are used in an amount of from 0.1 to 25% by weight, preferably 3 to 15% by weight and in particular 5 to 10% by weight.

### Compounds e)

As already stated, the compounds e) are foam stabilizers. In this regard, there are no restrictions per se. Preference is given to using substances which are selected from the group Sodium Laureth-4-Carboxylate, Glycereth-7, Laureth-7-Citrate (names in accordance with INCI nomenclature).

The compounds e) are used in an amount of from 0 to 10% by weight, preferably 0 to 7% by weight and in particular 0 to 5% by weight.

### EXAMPLES

### Substances Used

| | | |
|---|---|---|
| Nanocream | Emulsifiers from vegetable | (Sinerga) |
| | individual components | |
| Plantapon LGC | Plant-based APG emulsifier | (Cognis) |
| sorb | | |
| Plantapon ACG | Plant-based anionic | (Cognis) |
| | surfactant | |
| Plantapon LC 7 | Surfactant, citric acid | (Cognis) |
| | derivative | |
| Cognis 2006-G | Microcapsule dispersion with | (Cognis) |
| | menthol | |
| Myritol 318 | Caprylic acid/capric acid | (Cognis) |
| | triglyceride | |
| Phenonip | Preservative | (Clariant) |

### Pump Metering Device Used

Commercially available manually operable mechanical foam dispenser from Rexam Airspray International BV (bottle 200 ml HDPE—BE-43C1-200HE-10002; foamer pump white—F202.093.0001).

### Characterization of the Foam

Foam density (FD) is to be understood as meaning the quotient of foam weight (in grams) and foam volume (in liters) at a temperature of 20° C. The FD is given in g/l.

Foam decomposition time (FT) is to be understood as meaning the time in which, at 20° C., from a foam volume of 1 liter, the liquid present therein has flowed out in an amount of 50 ml. The FT is a measure of the durability of the foam. The FT is determined as follows: a graduated conical 1 liter sedimentation funnel (in accordance with DIN 12672-K) is filled with foam up to the 1000 ml mark over the course of 60 s. The foam is produced here from the aqueous emulsion and/or dispersion by means of the aforementioned pump foam bottle from Rexam Airspray International BV. A stopwatch is then used to measure the time in which 50 ml of liquid settle out.

### Assessment of the Application and Incorporability of the Foam

The assessment of the foam for its suitability for reloading was carried out as follows:

The spin-damp textile with a moisture content of 50±20% was supplied with foam. The number of foam displacements was governed by the dry weight of the textile and the weight of a foam displacement. 10%±5% foam weight was the aim, based on the dry textile. The foam displacements were spread uniformly on the textile and then massaged into the textile by hand. For this, the foam should be sufficiently stable and not penetrate too rapidly into the textile. The foam was assessed according to the following school grading system in accordance with the following evaluation scale:

| Grade | Definitions of the foam qualities |
|---|---|
| 1 | Stable, finely pored foam whose foam properties are |
| | optimally matched to uniform application. |
| 2 | Stable, finely pored and voluminous foam which only |
| | penetrates into the fabric following uniform |
| | distribution. |
| 3 | Stable, finely pored foam but which has a tendency to |
| | penetrate somewhat too quickly into the fabric. |
| 4 | Although foam is finely pored, it penetrates rapidly |
| | into the fabric. |
| 5 | Large-bubbled, aqueous foam which decomposes rapidly on |
| | the fabric and cannot be distributed uniformly |
| 6 | Foam decomposes immediately upon contact with the |
| | fabric. Distribution impossible. Spotting!! |

For the purposes of the present invention, foams with grades 1 to 4 are suitable for the reloading of textiles, whereas foams with grades 5 and 6 are not.

### WORKING EXAMPLES

### Example 1

### Pump Foam Based on Microcapsules

20.0 g of the microcapsule dispersion Cognis 2006-G (component b)) were initially introduced. After adding 20 g of completely demineralized water (component f)), the mixture was homogenized for 15 minutes. The mixture was heated to 50° C. The following surfactants T1, T2 and T3 were then stirred in in the following order:

T1) 7.0 g of Plantapon LGC sorb (foam-producing surfactant, component d)),
T2) 2.0 g of Plantapon ACG 35 (foam-stabilizing surfactant, component e)), and
T3) 1.0 g of Plantapon LC 7 (foam-stabilizing surfactant, component e)).

The mixture was homogenized for 5 minutes by stirring using a paddle stirrer. A further 50.0 g of demineralized water (component f)) were then added in portions. The mixture was heated to 80° C., thereafter stirred for 30 minutes and finally left to cool to room temperature. Finally, it was then filtered over a 250μ screen bag.

The pump foam was characterized as follows:

| | | |
|---|---|---|
| Brookfield viscosity at 25° C.: | 30 | mPas |
| pH: | 4.8 | |
| Foam density (FD) | 119 | g/l |
| Foam decomposition time (FT) | 15 | min |

### Examples 2 to 10

Example 1 was repeated. Instead of the total amount of surfactants T1, T2 and T3 of 10.0 g (=sum T1+T2+T3) used in example 1, the total amounts of the same surfactants T1, T2 and T3 which can be found in the table below were used. The weight ratio of the surfactants T1, T2 and T3 is given in each case in brackets. If a total amount of surfactants T1, T2 and T3 different from example 1 was used, the amount of water was amended so that the sum of all of the components in the pump foam formula (as in example 1) was 100 g.

The composition of examples 2 to 10 and the characterization of the foams can be found in the table below. Example 1 was also included in this table.

Table for examples 1 to 10:

| Surfactants¹⁾ | | | | | |
|---|---|---|---|---|---|
| (amount and | | Foam | Foam | | Application |
| ratio | | density | decomposition | | to textile |
| relative to | | (FD) | time (FT) | Foam | 1 = very good |
| one another) | Ex.²⁾ | (g/l) | (minutes) | quality | 6 = unusable |
| 1 (7-2-1) | 2 | 310 | 2.0 | wet, large- | 6 |
| | | | | bubbled, | |
| | | | | thin-liquid | |
| 3 (7-2-1) | 3 | 173 | 8.0 | wet, large- | 4 |
| | | | | bubbled, | |
| | | | | thin-liquid | |
| 7 (7-2-1) | 4 | 120 | 16.0 | wet, large- | 3 |
| | | | | bubbled, | |
| | | | | thin-liquid | |
| 10 (7-2-1) | 1 | 119 | 15.0 | voluminous, | 2 |
| | | | | finely | |
| | | | | pored foam | |
| 20 (7-2-1) | 5 | 114 | 18.0 | voluminous, | 4 |
| | | | | finely | |
| | | | | pored foam | |
| 10 (3.4-3.3-3.3) | 6 | 122 | 15.0 | somewhat | 2 |
| | | | | bubbly, | |
| | | | | stable | |
| 10 (1-2-7) | 7 | not | cannot be | liquid | cannot be |
| | | pumpable | determined | | determined |
| 5 (0.5-1-3.5) | 8 | not | cannot be | aqueous | 5 |
| | | pumpable | determined | foam | |
| 10 (1-7-2) | 9 | not | cannot be | liquid | cannot be |
| | | pumpable | determined | | determined |
| 5 (0.5-3.5-1) | 10 | 153 | 13.0 | liquid | 4 |

¹⁾Total amount of the surfactants T1 + T2 + T3 in g; the weight ratio of the surfactants T1:T2:T3 is given in brackets
²⁾Examples 1, 3 to 6 and 10 are in accordance with the invention; examples 2 and 7 to 9 serve for comparison.

### Example 11

### Pump Foam Based on a Cosmetic Oil

10.0 g of Myritol 318 (cosmetic oil, component a)), 7.5 g of Nanocream (emulsifier and foaming surfactant, therefore components c) and d)) and 1.0 g of Phenonip (preservative) were weighed into a beaker and stirred well. The mixture (oil phase) was then heated to 60° C. with stirring (dissolver disks or propeller stirrer at 150-300 rpm). At 60° C., the oil phase was homogeneous and virtually clear.

Then, water (component f)), which had been preheated to a temperature of 60° C., was added in three portions P1, P2 and P3 by dropping in. Throughout the entire time of the metered addition of the water, the temperature of the system was kept at 60° C.

The portion P1 (8.0 g) was slowly added dropwise. Following the addition of P1, a viscous intermediate phase was formed which was stirred for a further 5 minutes at 60° C.

The portion P2 (12.0 g) was likewise added dropwise. During this, the mixture became increasingly milky and more thin-liquid.

The portion P3 (61.5 g) was then stirred in relatively quickly.

Finally, the mixture was afterstirred for 5 minutes and then left to cool slowly to below 35° C.

The mixture obtained was then filtered through a fine-mesh screen (80μ) in order to ensure troublefree functional capability of the pump foam bottles used.

The pump foam was characterized as follows:

| | | |
|---|---|---|
| Brookfield viscosity at 25° C.: | 15 | mPas |
| pH: | 7.7 | |
| Foam density (FD) | 93 | g/l |
| Foam decomposition time (FT) | 8.5 | min |

### Examples 12 to 15

Example 11 was repeated. Instead of the total amount of Nanocream used in example 11, the amount of Nanocream which can be found in the table below was used. Since in each case an amount of Nanocream different from example 11 was used, the total amount of water was amended such that the sum of all of the components in the pump foam formula (as in example 11) was 100 g.

The composition of examples 12 to 15 and the characterization of the foams can be found in the table below. Example 11 was also included in this table.

Table for examples 11 to 15:

| | | | | | Application |
|---|---|---|---|---|---|
| | | Foam | Foam | | to textile |
| | | density | decomposition | | 1 = very |
| | | (FD) | time (FT) | Foam | good |
| Nanocream | Ex.¹⁾ | (g/l) | (minutes) | quality | 6 = unusable |
| 5.0 | 12 | 81 | 7.5 | relatively | 4 |
| | | | | finely | |
| | | | | pored | |
| 7.5 | 11 | 93 | 8.5 | finely | 3 |
| | | | | pored, | |
| | | | | stable | |
| 10.0 | 13 | 97 | 10.0 | stable | 3 |
| 15.0 | 14 | 130 | 14.0 | stable | 2 |

| | | | |
|---|---|---|---|
| 30.0 | 15 | cannot be determined since foam | not |
| | | is not pumpable | determined |

¹⁾Examples 11 to 14 are in accordance with the invention; example 15 serves for comparison.

What is claimed is:

1. (canceled)

2. A method for the reloading of textiles comprising:
1) providing an aqueous emulsion and/or dispersion comprising:
a) 0-25% by weight of one or more skincare oils,
b) 0-25% by weight of microcapsules loaded with active ingredients,
c) 0-20% by weight of one or more emulsifiers,
d) 0.1-25% by weight of one or more foam-producing agents,
e) 0-10% by weight of one or more foam stabilizers and
f) remainder to 100% by weight of water,
with the proviso that at least one of components a) and b) must be present in an amount of more than 0% by weight;
2) converting said emulsion and/or dispersion to foam form, said foam having a
foam density in the range of from 50 to 300 g/l, and a
foam disintegration time in the range of from 2 to 30 min. and
3) applying said foam to a textile.

3. A system comprising a manually operable foam dispenser and an aqueous emulsion and/or dispersion comprising:
a) 0-25% by weight of one or more skincare oils,
b) 0-25% by weight of microcapsules loaded with active ingredients,
c) 0-20% by weight of one or more emulsifiers,
d) 0.1-25% by weight of one or more foam-producing agents,
e) 0-10% by weight of one or more foam stabilizers and
f) remainder to 100% by weight of water,
with the proviso that at least one of components a) and b) must be present in an amount of more than 0% by weight,
and wherein the foam which emerges upon operating the foam dispenser has a
foam density in the range of from 50 to 300 g/l, and a
foam disintegration time in the range of from 2 to 30 min.

4. A system comprising a compressed-gas-operated foam dispenser and an aqueous emulsion and/or dispersion comprising:
a) 0-25% by weight of one or more skincare oils,
b) 0-25% by weight of microcapsules loaded with active ingredients,
c) 0-20% by weight of one or more emulsifiers,
d) 0.1-25% by weight of one or more foam-producing agents,
e) 0-10% by weight of one or more foam stabilizers and
f) remainder to 100% by weight of water,
with the proviso that at least one of components a) and b) must be present in an amount of more than 0% by weight,
and wherein the foam which emerges upon operating the foam dispenser has a
foam density in the range of from 50 to 300 g/l, and a
foam disintegration time in the range of from 2 to 30 min.
